(19) United States
(12) Patent Application Publication
(10) Pub. No.: US 2017/0215740 A1
(43) Pub. Date: Aug. 3, 2017

(54) PHOTOACOUSTIC APPARATUS, SUBJECT INFORMATION ACQUISITION METHOD, AND PROGRAM

(71) Applicant: CANON KABUSHIKI KAISHA, Tokyo (JP)

(72) Inventors: Takao NAKAJIMA, Kawasaki-shi (JP); Toshinobu TOKITA, Yokohama-shi (JP); Kazuhito OKA, Tokyo (JP)

(21) Appl. No.: 15/515,356

(22) PCT Filed: Sep. 26, 2015
(86) PCT No.: PCT/JP2015/004896

§ 371 Date: Mar. 29, 2017

(30) Foreign Application Priority Data
Sep. 30, 2014 (JP) 2014-201812

(51) Int. Cl. A61B 5/00; G01N 21/25; G01N 21/17

(52) U.S. Cl. CPC A61B 5/0095; G01N 21/1702; G01N 21/255

(57) ABSTRACT

A photoacoustic apparatus according to the present invention includes a light source, a conversion element configured to receive photoacoustic waves generated from light from the light source irradiated to a subject and output a signal, a color information acquiring unit configured to acquire information regarding color on the subject, a coefficient information acquiring unit configured to acquire information regarding an optical coefficient of the subject corresponding to the information regarding color of the subject acquired by the color information acquiring unit, and a subject information acquiring unit configured to acquire subject information based on the signal output from the conversion element and the information regarding the optical coefficient acquired by the coefficient information acquiring unit.

### TECHNICAL FIELD

The present invention relates to a photoacoustic apparatus using photoacoustic waves generated from absorbed light.

### BACKGROUND ART

A photoacoustic imaging technology is one of imaging technologies using light. In photoacoustic imaging, pulsed light generated from a light source is irradiated to a subject. The irradiated light propagates and diffuses within the subject, and energy of the light is absorbed at a plurality of locations from which photoacoustic waves occur. The photoacoustic waves are received by a transducer, and the received signal is analyzed within a processing device so that information regarding an optical characteristic value of the inside of the subject may be acquired as image data. This allows visualization of a distribution of optical characteristic values of the inside of the subject.

A sound pressure (hereinafter, called an initial sound pressure) P₀ occurring from photoacoustic waves generated by a light absorber within a subject can be represented by the following expression (1):

P₀=Γμₐ*Φ (1)

Here, Γ is a Gruneisen coefficient which is acquired by dividing a product of a volumetric expansion coefficient β and a square of the velocity of sound c by a specific heat Cₚ at constant pressure. Φ is a light fluence (which is a fluence of light reaching an absorbent) at a certain position (local region).

The initial sound pressure P₀ may be acquired by using a reception signal (PA signal) output from a probe which receives photoacoustic waves.

It is known that a Gruneisen coefficient takes a substantially constant value in accordance with a tissue. Thus, measurement and analysis of time variations of the PA signal at a plurality of locations may provide a product of an optical-absorption coefficient μₐ and a light fluence Φ, that is, a light energy absorption density.

As understood from Expression (1), in order to acquire subject information such as an optical-absorption coefficient μₐ of inside of a subject, the light fluence Φ of light reaching positions within a subject may be required to correct the light energy absorption density.

For example, in a case where it is assumed that light is irradiated to a large region with respect to the thickness of a subject and the light propagates like plane waves within the subject, the light fluence Φ may be represented by the following expression (2):

Φ=Φ₀*exp(−μ_{eff}*d) (2)

Here, Φ₀ is a fluence of light incident on a subject, μ_{eff} is an effective attenuation coefficient of the subject, d is a distance from the light incident position to a target position or the depth to the position of a light absorber.

As disclosed in NPL 1, a light transport equation or optical diffusion equation based on an optical-absorption coefficient μₐ^{ave} and a reduced scattering coefficient μₐ′ of a subject may be calculated by a numerical calculation technique to acquire a light fluence Φ. In other words, in order to acquire subject information such as an optical-absorption coefficient, an optical coefficient of a subject is required to be acquired.

### CITATION LIST

### Patent Literature

PTL 1: Japanese Patent No. 5713356

### Non Patent Literature

NPL 1: Bin Luo and Sailing He, OpticsExpress, Vol. 15, Issue 10, pp. 5905-5918 (2007)

### SUMMARY OF INVENTION

### Solution to Problem

A photoacoustic apparatus according to the present invention includes a light source, a conversion element configured to receive photoacoustic waves generated from light from the light source irradiated to a subject and output a signal, a color information acquiring unit configured to acquire information regarding color on the subject, a coefficient information acquiring unit configured to acquire information regarding an optical coefficient of the subject corresponding to the information regarding color of the subject acquired by the color information acquiring unit, and a subject information acquiring unit configured to acquire subject information based on the signal output from the conversion element and the information regarding the optical coefficient acquired by the coefficient information acquiring unit.

Further features of the present invention will become apparent from the following description of exemplary embodiments with reference to the attached drawings.

### BRIEF DESCRIPTION OF DRAWINGS

FIG. 1 is a schematic diagram illustrating a configuration of a photoacoustic apparatus according to an embodiment.

FIG. 2 is a flowchart illustrating an example of a flow for acquiring subject information according to the embodiment.

FIG. 3 is a flowchart illustrating an example of a flow for acquiring information regarding color of a subject according to the embodiment.

FIG. 4 is a flowchart illustrating another example of a flow for acquiring information regarding color of a subject according to the embodiment.

FIG. 5 illustrates an exemplary color chart used in the embodiment.

### DESCRIPTION OF EMBODIMENTS

Embodiments of the present invention will be described with reference to drawings. Like numbers refer like parts throughout in principle, and the description will be omitted.

The term “photoacoustic wave” herein refers to an acoustic wave occurring from absorbed light and may sometimes be called a photoacoustic wave.

In descriptions according to the present invention, the term “information regarding color” refers to a value of a parameter defined in a colorimetric system which represents color quantitatively. According to the present invention, any colorimetric system may be used as long as it is a colorimetric system which can represent color quantitatively. For example, “information regarding color” may be a parameter defined in a colorimetric system such as an RGB colorimetric system, an XYZ colorimetric system, an xyY colorimetric system, a L*u*v colorimetric system, a L*a*b colorimetric system, and the Munsell color system. For example, in the Munsell color system, the “information regarding color” may be a parameter including a hue, a brightness and a color saturation. The “information regarding color” may be a spatial distribution of parameters representing colors.

In the description according to the present invention, the “information regarding an optical coefficient” is a concept including optical coefficients such as an optical-absorption coefficient, a light scattering coefficient, and a light attenuation coefficient. An optical coefficient of a subject may be at least one representative value (such as a mean value) of an optical-absorption coefficient μₐ, a reduced scattering coefficient μₛ′, and an effective attenuation coefficient μ_{eff} of a subject, assuming that the subject is a homogeneous medium. In other words, the optical coefficient of a subject may be a representative value (such as a mean value) of an optical coefficient acquired when the optical coefficient is constant independent of positions of a subject. The information regarding an optical coefficient may be distribution information describing a spatial distribution of values of the optical coefficient.

The “information regarding an optical coefficient” is a concept including a light attenuation rate based on the optical coefficient of a subject. By multiplying a light attenuation rate by the fluence of light incident on a subject, a light fluence distribution within the subject may be derived. The “information regarding an optical coefficient of a subject” is a concept including a light fluence within a subject based on the optical coefficient of the subject.

Subject information (property value information) acquired according to one embodiment of the present invention reflects a light energy absorptivity. In other words, the subject information is information acquired from an optical-absorption coefficient μₐ. More specifically, the subject information may be an optical-absorption coefficient itself or a concentration of a substance contained in a tissue. The information regarding a concentration of a substance may be an oxygen saturation, a total hemoglobin concentration, or an oxyhemoglobin or deoxyhemoglobin concentration, for example. Alternatively, it may be a glucose concentration, a collagen concentration, a melanin concentration, or a volume fraction of fat or water. The subject information may be distribution data of two-dimensional or three-dimensional subject information. The distribution data of the subject information may be generated as image data.

The concentration of a substance may be acquired from an optical-absorption coefficient acquired by a measurement using light having different wavelengths. In this case, an optical-absorption coefficient within a subject may be acquired for each wavelength, and a concentration of a substance may be acquired by using those values and a wavelength dependency inherent to the substance. Particularly, an oxygen saturation of the blood may be acquired based on concentrations of oxyhemoglobin HbO and deoxyhemoglobin Hb. In a case where light having two different wavelengths is used, an oxygen saturation SO₂ may be acquired by Expression (3).

$$\begin{matrix}[ Math . 1 ] & \\ {SO}_{2} ( r ) = \frac{[ {HbO}_{2} ]}{[ {HbO}_{2} ] + [ Hb ]} = \frac{\frac{{μ}_{a}^{λ 2} ( r )}{{μ}_{a}^{λ 1} ( r )} \cdot {ɛ}_{Hb}^{λ 1} - {ɛ}_{Hb}^{λ 2}}{( {ɛ}_{HbO}^{λ 2} - {ɛ}_{Hb}^{λ 2} ) - \frac{{μ}_{a}^{λ 2} ( r )}{{μ}_{a}^{λ 1} ( r )} \cdot ( {ɛ}_{HbO}^{λ 1} - {ɛ}_{Hb}^{λ 1} )} & ( 3 )\end{matrix}$$

Here, μₐ^{λ1} represents an optical-absorption coefficient with a wavelength λ₁, and μₐ^{λ2} represents an optical-absorption coefficient with a wavelength λ₂. ε_{Hb0}^{λ1} represents a molar extinction coefficient of oxyhemoglobin with the wavelength λ₁, and ε_{Hb}^{λ1} represents a molar extinction coefficient of deoxyhemoglobin with the wavelength λ₁. ε_{Hb0}^{λ2} is a molar extinction coefficient of oxyhemoglobin with the wavelength λ₂, and ε_{Hb}^{λ2} represents a molar extinction coefficient of deoxyhemoglobin with the wavelength λ₂. ε_{Hb0}^{λ1}, ε_{Hb}^{λ1}, ε_{Hb0}^{λ2}, and ε_{Hb}^{λ2} are known values. r represents position coordinates.

A photoacoustic apparatus according to the following embodiment is mainly usable for a diagnosis and a follow-up study of a chemical treatment performed on a blood vessel disease or a malignant tumor of a human or an animal. Therefore, a subject may be a part of a living body or, more specifically, a test object in the skin, a subcutaneous region or the breast of a human or an animal. In particular, the subject is preferably a shallow region within several millimeter from a skin surface or a blood vessel within a dermal layer.

In a photoacoustic apparatus according to an embodiment, a light fluence distribution is required to be acquired in order to acquire subject information regarding an optical-absorption coefficient, as represented by Expression (1). Typically, in order to acquire a light fluence distribution, an optical coefficient of a subject is required to be acquired.

For example, in a case where a subject is a living body, the optical coefficient of a surface (skin) of a subject depends on the concentration of a structure substance such as a concentration of melanin or hemoglobin contained in the skin. The concentration of such a structure substance influences the color of the skin. For other subjects, the concentration of a structure substance of a subject influences the color of the subject.

Accordingly, the present inventor has found from those relationships that the information regarding an optical coefficient of a subject could be derived from the color of a subject.

A photoacoustic apparatus will be described below which acquires the information regarding color of a subject and acquires the information regarding an optical coefficient of the subject based on the information regarding color of the subject. With the photoacoustic apparatus according to this embodiment, the information regarding an optical coefficient of a subject may be easily acquired from information regarding color of the subject.

### Apparatus Configuration

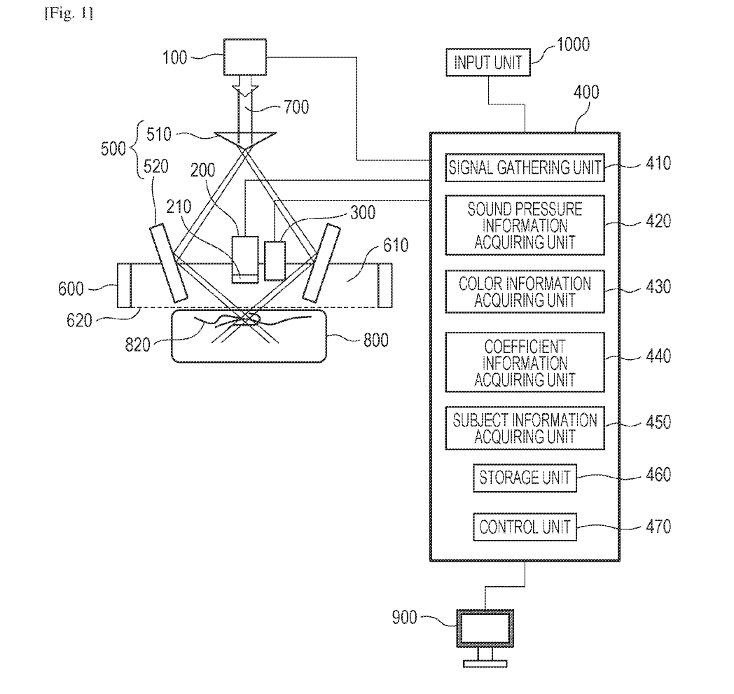

FIG. 1 is a schematic diagram illustrating a configuration of a photoacoustic apparatus according to an embodiment. The photoacoustic apparatus according to this embodiment includes a light source 100, a probe 200 including a conversion element 210, an image pickup unit 300, a processing unit 400, an optical system 500, a display unit 900, and an input unit 1000.

Pulsed light 700 from the light source 100 is guided by the optical system 500. The pulsed light 700 emitted from the optical system 500 is irradiated to a subject 800 and reaches a light absorber 820 within the subject 800. The light absorber 820 may typically be a blood vessel within a living body, particularly a substance such as hemoglobin or a tumor present within a blood vessel. The light absorber 820 absorbs energy of light and generates photoacoustic waves. The generated photoacoustic waves propagate within the subject and reach the conversion element 210.

The conversion element 210 receives the photoacoustic waves and thus outputs time-series reception signals. The reception signals output from the conversion element 210 are sequentially input to the processing unit 400. According to this embodiment, the conversion element 210 (receiving surface) in probe 200 is soaked in water 610 functioning as an acoustic matching material within a water tank 600. Thus, acoustic matching may be attempted between the subject 800 and the conversion element 210.

The processing unit 400 uses the input time-series reception signals to generate subject information. The processing unit 400 then transmits the generated data on the subject information to the display unit 900 and causes the display unit 900 to display an image and a numerical value of the subject information.

Component blocks of the photoacoustic apparatus according to this embodiment will be described.

### Light Source 100

The light source 100 may be a pulsed light source capable of radiating pulsed light of nano to micro second-order. The specific pulse width may be in a range of approximately 1 to 100 nanoseconds. The wavelength may be in a range of approximately 400 nm to 1600 nm. Particularly, light having a wavelength in a visible light region (equal to or higher than 400 nm and equal to or lower than 700 nm) is preferable for imaging a blood vessel near a surface of a living body at a high resolution. On the other hand, light having a wavelength (equal to or higher than 700 nm and equal to or lower than 1100 nm) which is less absorbed by a background tissue of the living body is preferable for imaging a deep part of a living body. However, terahertz wave, microwave, and radio wave regions may also be usable.

The light source 100 may specifically be a laser. For a measurement using light having a plurality of wavelengths, a laser may he used in which the wavelength of light oscillated by the laser can be converted. In a case where light with a plurality of wavelengths is irradiated to the subject 800, a plurality of lasers which oscillate light with different wavelengths from each other may be used by switching the oscillation wavelength or by irradiating alternately. A plurality of lasers may be handled collectively as a light source.

Various lasers may be used such as a solid laser, a gas laser, a dye laser, a semiconductor laser. Particularly, a pulsed laser may be used such as an Nd:YAG laser and an alexandrite laser. Alternatively, a Ti:sa laser or OPO (Optical Parametric Oscillators) laser excited by Nd:YAG laser light may be used. A light emitting diode may be used instead of such a laser.

### Probe 200

The probe 200 includes one or more conversion elements 210 and a housing. The conversion element 210 may be any conversion element capable of receiving acoustic waves and converting them to an electric signal, such as a piezoelectric element applying a piezoelectric phenomenon of lead zirconate titanate (PZT), for example, a conversion element applying oscillation of light, and a capacitance type conversion element such as a CMUT. In a case where a plurality of conversion elements 210 are provided, they may be arranged within a plane or a curved surface, as in a 1D array, a 1.5D array, a 1.75D array, or a 2D array, for example.

In order to acquire subject information in a wide range, the probe 200 may be configured to mechanically move about the subject 800 by using a scanning mechanism (not illustrated). The optical system 500 (from which the pulsed light 700 is irradiated) and the probe 200 may move in synchronization.

In a case where the probe 200 is of a hand-held type, the probe 200 has a grip which allows a user to grip the probe 200. In a case where the photoacoustic apparatus is a photoacoustic microscope, the probe 200 may be a focus type probe. In other words, an acoustic lens may be provided on the receiving surface of the conversion element 210. In a case where the photoacoustic apparatus is a photoacoustic tomography apparatus, the probe 200 may have a plurality of conversion elements 210.

The probe 200 may internally have an amplifier which amplifies an analog signal output from the conversion element 210.

### Image Pickup Unit 300

The image pickup unit 300 captures an optical image of a surface (such as the skin) of a subject 800. The image pickup unit 300 may be an optical camera such as a CCD camera and a CMOS camera. In other words, the image pickup unit 300 includes a light detecting unit and an image generating unit. The light detecting unit detects a reflected light from a surface of a subject and may be a CCD or a CMOS, for example. The light generating unit generates an optical image based on a detection signal output from the light detecting unit. The light detecting unit may be any element capable of detecting light and outputting an electric signal. The image generating unit may be a processor such as a CPU and a GPU (graphics processing unit) or an operation circuit such as an FPGA (field programmable gate array) chip. The image generating unit may include a plurality of processors and operation circuits instead of one processor or one operation circuit. The functions to be performed by the image generating unit may also he performed by the processing unit 400.

The image pickup unit 300 may include a light source for reducing differences in illumination environments for imaging operations. Thus, the light detecting unit could detect reflected light from the light source provided in the image pickup unit 300. The light source included in the image pickup unit 300 may be a light source which radiates white light including a wide wavelength. For example, the light source included in the image pickup unit 300 may be an LD, an LED or a fluorescent light which radiates white light.

### Processing Unit 400

Next, a configuration within the processing unit 400 according to this embodiment will be described. The processing unit 400 according to this embodiment includes a signal gathering unit 410, a sound pressure information acquiring unit 420, a color information acquiring unit 430, a coefficient information acquiring unit 440, a subject information acquiring unit 450, a storage unit 460, and a control unit 470.

The signal gathering unit 410 gathers time-series analog reception signals output from the conversion element 210 for each channel and performs signal processing including amplification of reception signals, AD conversion of analog reception signals, and storage of digitized reception signals. The signal gathering unit 410 may be a circuit generally called a DAS (Data Acquisition System). More specifically, the signal gathering unit 410 includes an amplifier which amplifies a reception signal and an AD converter which digitizes an analog reception signal.

The sound pressure information acquiring unit 420 acquires sound pressure information generated at different positions within a subject by using reception signals output from the signal gathering unit 410. Hereinafter, the sound pressure information generated at different positions within a subject will also be called an initial sound pressure distribution within a subject.

The color information acquiring unit 430 acquires information regarding color of a subject 800 based on an optical image of a surface of the subject 800 captured by the image pickup unit 300 and a color designated by a user.

The coefficient information acquiring unit 440 acquires information regarding an optical coefficient of a subject 800 based on information regarding color of the subject 800 acquired by the color information acquiring unit 430.

The subject information acquiring unit 450 calculates subject information such as an absorption coefficient distribution from sound pressure distribution data acquired by the sound pressure information acquiring unit 420 and information regarding an optical coefficient of the subject acquired by the coefficient information acquiring unit 440.

Each of the sound pressure information acquiring unit 420, color information acquiring unit 430, coefficient information acquiring unit 440, and subject information acquiring unit 450 may be a processor such as a CPU and a CPU (graphics processing unit) or an operation circuit such as an FPGA (field programmable gate array) chip. Each of them may include a plurality of processors and operation circuits instead of one processor or one operation circuit.

The storage unit 460 may store reception signals having undergone an AD conversion process, distribution data, display image data, and measurement parameters. The storage unit 460 may further store a relationship table or a relational expression, which will be described below, representing a relationship between information formation regarding color and information regarding an optical coefficient. The storage unit 460 may further store a process to be performed by a subject information acquisition method as a program to be executed by a control unit within the processing unit 400. The storage unit 460 storing a program is a nontemporary printing medium. The storage unit 460 may typically be one of storage media such as a FIFO memory, a ROM, a RAM, and a hard disk. The storage unit 460 may include a plurality of storage media instead of one storage medium.

The processing unit 400 further includes a control unit 470 for controlling operations of the components of the photoacoustic apparatus. The control unit 470 supplies control signals and data necessary for the components of the photoacoustic apparatus via a bus. More specifically, the control unit 470 may supply a light emission control signal which instructs light emission to the light source 100, a reception control signal for the conversion element 210 within the probe 200, and a control signal for a scanning mechanism. The control unit 470 may typically be a CPU.

### Optical System 500

The optical system 500 conducts light from the light source 100 to the subject 800. The optical system 500 may be an optical element such as a lens, a mirror, and an optical fiber. In a biological information acquiring apparatus for inspecting a breast, for example, a light emitting unit in the optical system 500 may irradiate beam light having a wider diameter through a lens. On the other hand, in a photoacoustic microscope, the light emitting unit in the optical system 500 may include a lens for a higher resolution and may irradiate a light beam with a narrow diameter. The optical system 500 may be moved about the subject 800, which allows wide-range imaging of the subject 800. Without using the optical system 500, light may be radiated directly from the light source 100 to the subject 800.

According to this embodiment, the optical system 500 may be an axicon lens 510, and an optical mirror 520. The axicon lens 510 is disposed to conduct the pulsed light 700 radiated from the light source 100 in a ring shape and not to irradiate the pulsed light 700 to the probe 200. The optical mirror 520 is disposed to collect the ring-shaped pulsed light 700 to a target position on the subject 800.

### Water Tank 600

The water tank 600 is a container in which water 610 as an acoustic matching material can be filled. According to this embodiment, the conversion elements 210 included in the probe 200 are soaked in water 610. This allows acoustic matching through the water between the subject 800 and the conversion element 210. A surface 620, indicated by a broken line in FIG. 1, in contact with the subject 800 may be a thinner film than a wavelength of a photoacoustic wave to easily allow photoacoustic waves to pass through. More preferably, the contact surface 620 may have a ¼ thickness of the wavelength of a photoacoustic wave. The acoustic matching material and the contact surface 620 may contain a material which does not absorb the pulsed light 700 easily. For example, the acoustic matching material may be water or alcohol, and the contact surface 620 may contain polyethylene, for example.

### Subject 800

Though the subject 800 is not a component of the photoacoustic apparatus of the present invention, the subject 800 will be described below. The photoacoustic apparatus according to this embodiment is mainly usable for a diagnosis and a follow-up study of a chemical treatment performed on a blood vessel disease or a malignant tumor of a human or an animal. Therefore, the subject 800 may be a living body, and, more specifically, it may be a diagnosis target region such as the breast, the neck, or the abdomen of a human or animal body.

The light absorber 820 within the subject 800 may have a relatively high optical-absorption coefficient within the subject 800. For example, in a case where a human body is a measurement target, the target of the light absorber 820 may be oxyhemoglobin or deoxyhemoglobin or a blood vessel containing a large amount of them or a malignant tumor containing many neovessels. Alternatively, the target may be carotid artery plaque.

### Display Unit 900

The display unit 900 functioning as a display unit may be a display such as an LCD (liquid crystal display), a CRT (cathode ray tube), and an organic EL display. The display unit 900 may he separately provided and be connected to the photoacoustic apparatus instead of being included in the photoacoustic apparatus of this embodiment.

### Input Unit 1000

The input unit 1000 is a member configured to allow a user to input desired information. Information input by a user is transmitted from the input unit 1000 to the processing unit 400. The input unit 1000 may be a keyboard, a mouse, a touch panel, a dial, or a button. In a case where the input unit 1000 is a touch panel, the display unit 900 may be a touch panel also usable as the input unit 1000. The input unit 1000 may be separately provided from the photoacoustic apparatus of the present invention.

### Subject Information Acquisition Method

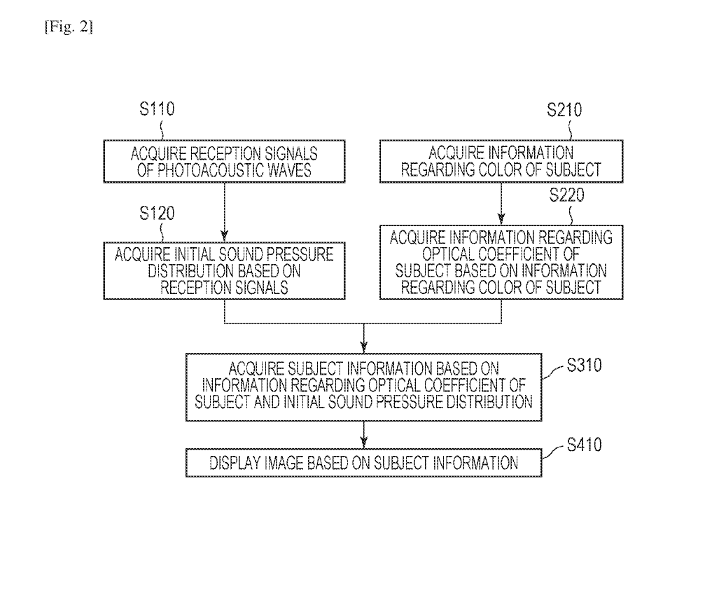

Next, a flow for acquiring subject information by the photoacoustic apparatus according to this embodiment will be described with reference to FIG. 2. The control unit 470 reads out a program in which a subject information acquisition method is described from the storage unit 460 to cause the photoacoustic apparatus to execute the following subject information acquisition method.

### S110: Step of Acquiring Reception Signal of Photoacoustic Waves

In this step, light generated by the light source 100 is irradiated as pulsed light 700 to a subject 800 through the optical system 500. The pulsed light 700 is absorbed by the subject 800, and a photoacoustic effect thereof causes photoacoustic waves therefrom. The conversion elements 210 receive the photoacoustic waves and output time-series analog reception signals. The signal gathering unit 410 gathers the time-series analog reception signals output from the conversion elements 210 for each channel and may perform amplification processing on the reception signals and AD conversion processing on the analog reception signals. The signal gathering unit 410 stores the digitized reception signals in the storage unit 460. The time-series reception signal data stored in the storage unit 460 may also be called photoacoustic data. According to the present invention, the term “reception signal” refers to a concept including an analog signal and a digital signal.

### S120: Step of Acquiring Initial Sound Pressure Distribution Within Subject Based on Reception Signal

In this step, the sound pressure information acquiring unit 420 acquires sound pressure information of photoacoustic waves at different positions within a subject based on reception signals output from the signal gathering unit 410. In other words, the sound pressure information acquiring unit 420 acquires an initial sound pressure distribution within the subject 800.

In a case where the photoacoustic apparatus is a photoacoustic tomography apparatus, the sound pressure information acquiring unit 420 reconstructs an image by using reception signals acquired for each channel so that data on an occurring sound pressure corresponding to a position on a two-dimensional or three-dimensional space coordinate system. For the image reconstruction, the sound pressure information acquiring unit 420 may apply Universal Back Projection (UBP) disclosed in PLT 1 or a publicly known reconstruction scheme such as Filtered Back Projection (FBP). As an alternative reconstruction scheme, the sound pressure information acquiring unit 420 may apply phasing addition (Delay and Sum) processing.

Alternatively, the sound pressure information acquiring unit 420 may perform envelop detection on the acquired reception signals with tune changes, then convert the time axis direction in the signal for each light pulse to a depth direction and plot the results in the space coordinate system. The sound pressure information acquiring unit 420 performs this for each scan position to acquire sound pressure distribution data. Particularly in a case where the photoacoustic apparatus is a photoacoustic microscope, this scheme may be used.

### S210: Step of Acquiring Information Regarding Color of Subject

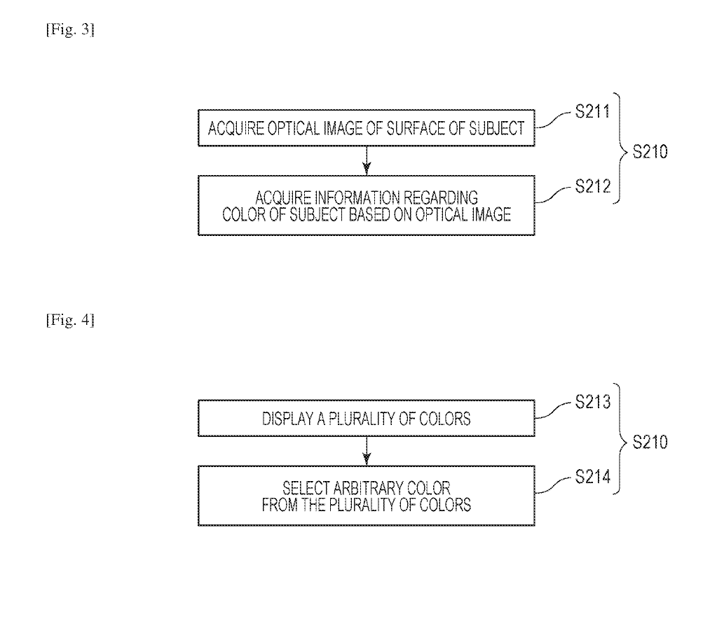

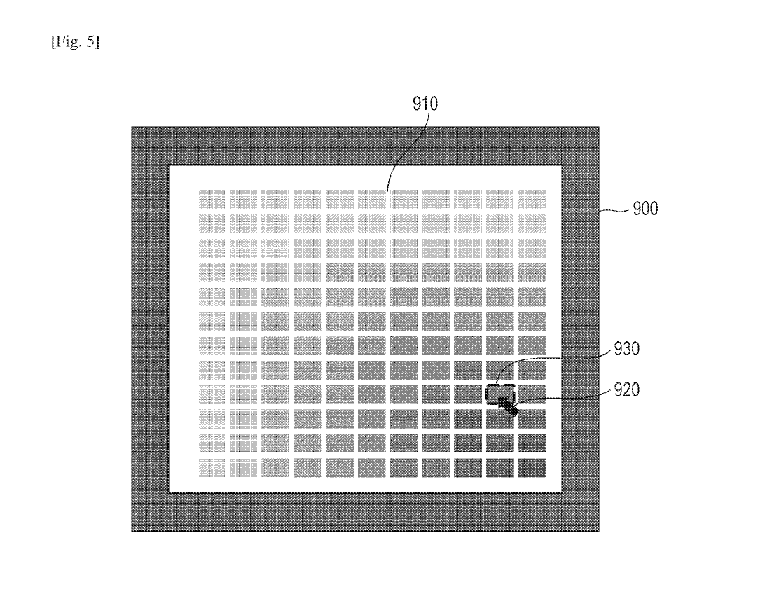

In this step, the color information acquiring unit 430 acquires information regarding color of the subject 800 and stores it in the storage unit 460. An example of a specific flow of this step will be described below with reference to FIGS. 3 and 4.

First of all, an example of a flow for acquiring information regarding color of the subject 800 from an optical image by the color information acquiring unit 430 will be described with reference to FIG. 3.

### S211: Step of Acquiring Optical Image

The image pickup unit 300 acquires an optical image of a surface of the subject 800. The data on the acquired optical image of the surface of the subject 800 are stored in the storage unit 460.

### S212: Step of Acquiring Information Regarding Color Based on Optical Image

The color information acquiring unit 430 may analyze an optical image of the subject 800 acquired in S211 and thus acquire information regarding color of the subject 800.

For example, the color information acquiring unit 430 is capable of acquiring information regarding color in one pixel of an optical image as information regarding color at positions of the subject 800. For example, information regarding color at each position of the subject 800 may be acquired based on information of one pixel of a central point of the optical image.

The color information acquiring unit 430 may acquire an average value of information regarding color at a plurality of pixels within an optical image as information regarding color at positions of the subject 800.

The color information acquiring unit 430 may also acquire information regarding a color with the highest frequency value from a data set of the information regarding color within an optical image as information regarding color at positions of the subject 800.

The color information acquiring unit 430 may cause the display unit 900 to display an optical image, and a user may input arbitrary coordinates on the optical image by using the input unit 1000. The color information acquiring unit 430 may acquire information regarding color corresponding to the coordinates output from the input unit 1000 as information regarding color of the subject 800. For example, a user may manipulate an icon on the display unit 900 by using the input unit 1000 to designate arbitrary coordinates on the optical image displayed on the display unit 900. A user may input an arbitrary area on an optical image by using the input unit 1000, and the coordinates of the input area may be output. In this case, an arbitrary area which may be input by using the input unit 1000 is information regarding coordinates of the optical image.

Next, an example of a flow for inputting information regarding color of the subject 800 by a user by using the input unit 1000 will be described with reference to FIG. 4.

### S213: Step of Displaying Plurality of Colors

The color information acquiring unit 430 transmits data on a plurality of colors stored in the storage unit 460 to the display unit 900 and causes the display unit 900 to display the plurality of colors. The display unit 900 may display a plurality of colors in parallel or may sequentially change over and display a plurality of colors. For example, as illustrated in FIG. 5, the color information acquiring unit 430 causes the display unit 900 to display data on a color chart 910 stored in the storage unit 460.

S214: Step of Selecting Arbitrary Color from Plurality of Colors

A user uses the input unit 1000 to select an arbitrary color. For example, as illustrated in FIG. 5, a user manipulates an arrow icon 920 displayed on the display unit 900 by using the input unit 1000 to select a color icon 930 corresponding to a desired color from the color chart 910 and select the arbitrary color. In FIG. 5, the selected color icon 930 of a plurality of color icon included in the color chart 910 is illustrated within a dashed-line frame.

It may be configured to allow a user to select an arbitrary color from a color chart provided separately from the display unit 900 and allow the input unit 1000 to input the color. In this case, the processing in S214 may be executed by skipping the processing in S213. In this step, any method may be applied as long as a user is allowed to input color of a subject by using the input unit 1000. In other words, the input unit 1000 may be configured to allow a user to select an arbitrary color.

The input unit 1000 may be configured to allow direct input of information regarding color. For example, a user may be allowed to input RGB values as information regarding color defined in an RGB colorimetric system by using the input unit 1000.

The color information acquiring unit 430 acquires information regarding color selected by a user and output from the input unit 1000 and stores it as information regarding color of the subject 800 in the storage unit 460.

Thus, a user may input a color of a subject intuitively, and the subject color information acquiring unit 450 may acquire information regarding an optical coefficient corresponding to the input color so that information regarding the color may easily be acquired.

In the processing in S210, instead on the examples illustrated in FIGS. 3 and 4, a color measurement apparatus as disclosed in Japanese Patent Laid-Open No. 2003-315154 may be applied to measure information regarding color.

### S220: Step of Acquiring Information Regarding Optical Coefficient of Subject Based on Color of Subject

In this step, the coefficient information acquiring unit 440 acquires information regarding an optical coefficient of the subject 800 based on the information regarding color of the subject 800 acquired in S210. Furthermore, in this step, the coefficient information acquiring unit 440 acquires information regarding an optical coefficient of the subject 800 based on a relationship table or relational expression, which is prepared in advance, representing a relationship between information regarding color and information regarding optical coefficients.

For example, the coefficient information acquiring unit 440 may read out from the relationship table stored in the storage unit 460 the information regarding an optical coefficient of the subject 800 corresponding to the information regarding color of the subject 800 acquired in S210.

Because the table stores discrete values, the information regarding a color acquired in S210 and values stored in the table may not be matched sometimes, in this case, the coefficient information acquiring unit 440 is capable of reading out from the table information regarding an optical coefficient of the subject 800 corresponding to a parameter value closest to the information regarding the color acquired in S210. Alternatively, the coefficient information acquiring unit 440 may calculate information regarding an optical coefficient of the subject 800 by interpolating a value in the table.

Alternatively, the coefficient information acquiring unit 440 may use the information regarding color of the subject 800 acquired in S210 to solve the relational expression stored in the storage unit 460 so that the information regarding an optical coefficient of the subject 800 may be acquired.

Next, an example of a method for generating a relationship table or a relational expression to be used in S220 will be described. An example will be described in a case where the subject 800 is a living body including an epidermis and a tissue deeper than the epidermis.

In order to generate a relationship table or relational expression to be used in S220, a phantom imitating the epidermis of the subject 800 may be used. A plurality of phantoms imitating the epidermis of the subject 800 may be produced by using a material containing various pigments at various concentrations and in various proportions. Because the epidermis of a living body 200 μm thick appropriately, the phantom is defined to have a thickness of 200 μm, for example.

Next, the information regarding color of a plurality of phantoms containing different types and concentrations of pigments is measured in accordance with the method described in the description of S210. In a case where the color of a phantom is different from the color of a surface of the subject because the phantom is thin, a structure may be produced in which a phantom imitating the epidermis and a tissue deeper than the epidermis are stacked. The information regarding color of the surface of the stacking structure may be acquired in accordance with the method described in the description of S210 as information regarding color of a phantom imitating the epidermis.

The information regarding optical coefficients of a plurality of phantoms imitating the epidermis is acquired. For example, the phantom is irradiated with light, and the intensity of the light having passed through the phantom is detected by a light detector such as a photodetector. Based on the detection signal and the incident intensity of the light irradiated to the phantom from the light detector, the light attenuation rates and optical-absorption coefficients corresponding to the phantoms imitating the epidermis may be acquired as the information regarding optical coefficients. The processing unit 400 may then generate a relationship table or a relational expression by associating information regarding colors of a plurality of phantoms and information regarding optical coefficients of the plurality of phantoms and store it in the storage unit 460.

The influence of scattering of light is ignorable near a surface of the subject 800 having a thickness smaller than the average free path (300 μm to 1000 μm appropriately when the subject is a living body). Therefore, in a case where the epidermis of a living body is the subject of the measurement, the light attenuation rate may be represented by Expression (4) based on the optical-absorption coefficient μₐ^{sur} of the epidermis.

Light Attenuation Rate On Surface Of Subject (Epidermis)=exp(−μₐ^{sur}*d) (4)

Based on the detection signal from the light detector and the incident intensity of light irradiated to the phantom, a light fluence distribution within the phantom imitating the epidermis may be acquired in accordance with Expression (4).

Without limiting to the method above, any method may be applied if the method allows acquisition of information regarding an optical coefficient of a phantom imitating the epidermis. A relationship table may be generated in which the acquired information regarding colors of the epidermis and the acquired information regarding the optical coefficients are associated and may be stored in the storage unit 460. Alternatively, a relational expression may be generated from the information regarding colors of the phantoms of the epidermis and the information regarding the optical coefficients and be stored in the storage unit 460. In the example above, a relationship table or a relational expression may be generated based on the information regarding an optical coefficient of an epidermis part of the subject 800 and the information regarding color of the subject 800.

In the example above, the 2-layer structure including the epidermis and a tissue deeper than the epidermis is handled as the subject 800. However, the subject 800 may be a medium having a constant optical characteristic. In this case, a plurality of phantoms having a constant optical characteristic are produced. By applying an existing optical coefficient measurement method to those phantoms, the optical coefficients of the phantoms are acquired. For example, in a case where the thickness of the phantoms is larger than the average free path, Time Resolved Spectroscopy may be applied to acquire an average optical-absorption coefficient and reduced scattering coefficient of the phantoms as optical coefficients. Phantoms having a constant optical characteristic may be stored in an optical cell, and the intensities of the transmitted light and reflected light of the irradiated light may be detected. Then, based on the intensities of the transmitted light and reflected light and the intensity of the incident light, an average optical-absorption coefficient and reduced scattering coefficient of phantoms may be acquired by applying Monte Carlo method, for example. Phantoms having a constant optical characteristic may be stored in a cuvette, and the intensities of the transmitted light of the light irradiated to the cuvette may he detected. Then, based on the intensity of the transmitted light and the intensity of the incident light, the light attenuation rate corresponding to each phantom and an effective attenuation coefficient of each phantom may be acquired in accordance with Expression (5).

Light Attenuation Rate Within Subject=exp(−μ_{eff}*d) (5)

Here, μ_{eff} is an effective attenuation coefficient of a subject, and d is a depth of a region of interest. Based on the intensity of transmitted light and the intensity of incident light, a light fluence distribution within a phantom may he acquired in accordance with Expression (2).

When the optical-absorption coefficient and reduced scattering coefficient of a phantom are acquired as optical coefficients, a light transport equation or optical diffusion equation as disclosed in NPL 1 may be solved by a numerical value calculation scheme to acquire a light fluence distribution within the phantom. The numerical value calculation scheme may be a finite element analysis and Monte Carlo method.

Instead of use of a phantom, an excised skin may be used to create the relationship table or relational expression. Those table generation methods are given for illustration purpose only, but it should be understood that the table generation method is not limited.

The processing in S210 to S220 may be performed after the processing in S110 to S120, or the processing in S110 to S120 may he performed after the processing in S210 to S220. At least one of S110 to S120 and at least one of S210 to S220 may be performed in parallel.

### S310: Step of Acquiring Information Based on Optical Coefficient of Subject and Initial Sound Pressure Distribution

In this step, the subject information acquiring unit 450 acquires subject information based on the initial sound pressure distribution of the subject 800 acquired in S120 and the information regarding the optical coefficients of the subject 800 acquired in S220. An example will be described below in which an optical-absorption coefficient distribution within the subject 800 is acquired as the subject information. In the following example, in a case where the subject 800 includes a medium having a constant optical characteristic, the optical-absorption coefficient distribution in an arbitrary area of the medium may be acquired as the optical-absorption coefficient distribution within the subject 800. Further in the following example, in a case where the subject 800 is a living body including the epidermis and a tissue deeper than the epidermis, the optical-absorption coefficient distribution of a skin part may typically be acquired as the optical-absorption coefficient distribution within the subject 800.

First of all, an example will be described in which an optical-absorption coefficient distribution within the subject 800 is acquired in a case where the information regarding an optical coefficient of the subject 800 is a light fluence distribution of the pulsed light 700 within the subject 800. In this case, the subject information acquiring unit 450 solves Expression (6) by using an initial sound pressure distribution P₀ acquired in S120 and a light fluence distribution Φ corresponding to the information regarding color of the subject 800 stored in the storage unit 460 to acquire an optical-absorption coefficient distribution μₐ.

$$\begin{matrix}[ Math . 2 ] & \\ {μ}_{a} = \frac{{P}_{0}}{Γ \cdot Φ} & ( 6 )\end{matrix}$$

Next, an example will be described in which an optical-absorption coefficient distribution is acquired in a case where the information regarding an optical coefficient of the subject 800 is an attenuation rate of light within the subject 800. As described above, an attenuation rate of light is a parameter which may provide a light fluence distribution within a subject by multiplying the fluence of light incident on the subject by it. In this case, the subject information acquiring unit 450 multiplies the fluence of light incident on the subject 800 by a light attenuation rate corresponding to the information regarding color of the subject 800 stored in the storage unit 460 to acquire the light fluence distribution Φ within the subject 800. The subject information acquiring unit 450 then uses the acquired light fluence distribution Φ to solve Expression (6) to acquire the optical-absorption coefficient distribution.

The fluence of light incident on the subject 800 could be grasped by reading out a set value for output from the light source 100 by the subject information acquiring unit 450 or detecting the intensity of the pulsed light 700 by a light detector such as a photodiode.

Next, an example will be described in which an optical-absorption coefficient distribution is acquired in a case where the information regarding an optical coefficient of the subject 800 is an optical coefficient of the subject 800. In this case, for example, the subject information acquiring unit 450 may use an effective attenuation coefficient of the subject 800 acquired as the information regarding an optical coefficient to solve Expression (2) for acquiring the light fluence distribution Φ. The subject information acquiring unit 450 may use the optical-absorption coefficient μₐ^{ave} and reduced scattering coefficient μₛ′ of the subject 800 acquired as the information regarding an optical coefficient in accordance with a method as disclosed in NPL 1 to acquire the light fluence distribution Φ. The subject information acquiring unit 450 uses the acquired light fluence distribution Φ to solve Expression (6) to acquire an optical-absorption coefficient distribution.

A case will be described where the subject 800 is a living body including the epidermis and a tissue deeper than the epidermis and where the region for which an optical-absorption coefficient is to be acquired is the tissue deeper than the epidermis. In this case, information regarding an optical coefficient corresponding to the information regarding color of the subject 800 may typically be considered as the information regarding an optical coefficient in the epidermis of the subject 800. Accordingly, the subject information acquiring unit 450 may acquire the intensity of light having passed through the epidermis of the subject 800, that is, the intensity of the light incident of the tissue deeper than the epidermis based on the information regarding an optical coefficient corresponding to the information regarding color of the subject 800. For example, a case will be discussed where the coefficient information acquiring unit 440 acquires a light attenuation rate in the epidermis as the information regarding an optical coefficient corresponding to the information regarding color of the subject 800. In this case, the subject information acquiring unit 450 uses the light attenuation rate of the epidermis and the intensity information of the light incident on the epidermis to acquire the intensity of light incident on the deeper tissue in accordance with Expression (5). Then, the intensity of the incident light may be defined as an initial condition to solve Expression (2) and a propagation equation such as a light transport equation and optical diffusion equation to acquire a light fluence distribution within the deeper tissue. The optical coefficient of the deeper tissue used in the calculation may be acquired by a publicly known method. By solving Expression (6) using the acquired light fluence distribution, the optical-absorption coefficient distribution within the deeper tissue may be acquired. Thus, the influence of the optical characteristic of the epidermis on the light fluence distribution of the deeper tissue could be easily reflected on the acquired light fluence distribution.

In other words, for a region at a predetermined depth or less from a surface of a subject, the subject information acquiring unit 450 may use the information regarding an optical coefficient corresponding to the information regarding color of the subject 800 to calculate the light fluence distribution. On the other hand, for a deep region at a depth more than the predetermined depth from the surface of the subject, the subject information acquiring unit 450 may the light fluence distribution calculated by using the information regarding an optical coefficient corresponding to the information regarding color of the subject 800 to calculate the light fluence distribution. The predetermined depth here may be equal to or less than 1000 μm appropriately corresponding to the average free path. The predetermined depth may be equal to or less than 300 μm appropriately corresponding to the average free path. The predetermined depth may be equal to or less than 200 μm appropriately corresponding to the thickness of the epidermis.

Having described that, according to this embodiment, the optical-absorption coefficient distribution is acquired as the subject information, for example, the subject information is not limited thereto. The aforementioned steps may be performed for each of a plurality of wavelengths of light to acquire an optical-absorption coefficient distribution of each of the wavelengths. Then, based on the distributions, spectrum information such as an oxygen saturation may be acquired as the subject information. Alternatively, the spectrum information may be acquired from the initial sound pressure distribution and light fluence distribution for each of the wavelengths without the optical-absorption coefficient distributions. For example, in a case where two measurement wavelengths are given, the subject information acquiring unit 450 is allowed to calculate the oxygen saturation from Expression (3). In a case where three or more measurement wavelengths are given, the subject information acquiring unit 450 is allowed to calculate the oxygen saturation by fitting the optical-absorption coefficient distribution of each of the measurement wavelengths by using the absorbance spectrum of oxyhemoglobin and deoxyhemoglobin.

### S410: Display Subject Information

The subject information acquiring unit 450 transmits the data of the subject information acquired in S310 to the display unit 900 and displays an image of the subject information on the display unit 900. The display unit 900 is capable of displaying information regarding the subject information such as a numerical value of the subject information, without limiting to an image of the subject information.

As described above, the photoacoustic apparatus according to this embodiment allows easy acquisition of the information regarding an optical coefficient of a subject based on the information regarding color of a subject. This also allows easy acquisition of the subject information by using the information regarding the optical coefficient of the subject.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-201812, filed Sep. 30, 2014, which is hereby incorporated by reference herein in its entirety.

1. A photoacoustic apparatus comprising:
a light source;
a conversion element configured to receive photoacoustic waves generated from light from the light source irradiated to a subject and output a signal;
a color information acquiring unit configured to acquire information regarding color on the subject;
a coefficient information acquiring unit configured to acquire information regarding an optical coefficient of the subject corresponding to the information regarding color of the subject acquired by the color information acquiring unit; and
a subject information acquiring unit configured to acquire subject information based on the signal output from the conversion element and the information regarding the optical coefficient acquired by the coefficient information acquiring unit.

2. The photoacoustic apparatus according to claim 1, further comprising an image pickup unit configured to acquire an optical image of the subject,
wherein the color information acquiring unit acquires the information regarding color of the subject based on the optical image of the subject acquired by the image pickup unit.

3. The photoacoustic apparatus according to claim 2, further comprising an input unit usable for inputting information regarding coordinates on an optical image of the subject displayed on a display unit,
wherein the color information acquiring unit acquires information regarding color on the optical image of the subject at the coordinates output from the input unit as the information regarding color of the subject.

4. The photoacoustic apparatus according to claim 1, further comprising an input unit configured to be usable for inputting information regarding color,
wherein the color information acquiring unit acquires the information regarding color output from the input unit.

5. The photoacoustic apparatus according to claim 1, further comprising an input unit configured to be usable for inputting an arbitrary color,
wherein the color information acquiring unit acquires the arbitrary information regarding color output from the input unit as information regarding color of the subject.

6. The photoacoustic apparatus according to claim 5, wherein the input unit is configured to be usable for selecting an icon corresponding to the arbitrary color from a plurality of icons representing a plurality of colors displayed on the display unit.

7. The photoacoustic apparatus according to claim 1, further comprising a storage unit configured to store a relationship table or a relational expression between information regarding color and information regarding an optical coefficient,
wherein the coefficient information acquiring unit acquires the information regarding an optical coefficient of the subject corresponding to the information regarding color of the subject acquired by the color information acquiring unit based on the relationship table or the relational expression.

8. The photoacoustic apparatus according to claim 1, wherein the subject information acquiring unit
acquires a light fluence distribution of a region at a predetermined depth or less from a surface of the subject by using the information regarding the optical coefficient; and
acquires a light fluence distribution of a region deeper than the predetermined depth from the surface of the subject by using the light fluence distribution of the region up to the predetermined depth or less.

9. The photoacoustic apparatus according to claim 8, wherein the predetermined depth is equal to or lower than 1000 μm.

10. The photoacoustic apparatus according to claim 8, wherein the predetermined depth is equal to or lower than 200 μm.

11. The photoacoustic apparatus according to claim 1, wherein the information regarding an optical coefficient is one of an optical-absorption coefficient, a light attenuation rate, and a light fluence distribution.

12. The photoacoustic apparatus according to claim 1, wherein the coefficient information acquiring unit acquires an optical coefficient of the subject as the information regarding the optical coefficient of the subject; and
the subject information acquiring unit
acquires a light fluence distribution within the subject based on the optical coefficient of the subject; and
acquires the subject information based on the signal a light fluence distribution within the subject.

13. The photoacoustic apparatus according to claim 12, wherein the subject information acquiring unit
acquires an initial sound pressure distribution within the subject based on the signal; and
acquires the subject information based on the initial sound pressure distribution within the subject and the light fluence distribution within the subject.

14. The photoacoustic apparatus according to claim 1, wherein the subject information acquiring unit acquires an optical-absorption coefficient distribution within the subject as the subject information.

15. The photoacoustic apparatus according to claim 1, further comprising a display unit configured to display information regarding the subject information.

16. An information processing method comprising:
acquiring information regarding color of a subject; and
acquiring information regarding an optical coefficient of the subject corresponding to the information regarding color of the subject.

17. The information processing method according to claim 16, further comprising acquiring subject information based on a signal acquired by receiving photoacoustic waves generated from light irradiated to the subject and the information regarding the optical coefficient.

18. A program causing a processing unit to execute the information processing method according to claim 16.
